(19) United States
(12) Patent Application Publication
(10) Pub. No.: US 2018/0327869 A1
(43) Pub. Date: Nov. 15, 2018

(54) A Method of Screening for Modulation of Cell Signalling Pathways

(71) Applicant: CAMBRIDGE ENTERPRISE LIMITED, Cambridge (GB)

(72) Inventor: Bryn Hardwick, Cambridge (GB)

(73) Assignee: CAMBRIDGE ENTERPRISE LIMITED, Cambridge (GB)

(21) Appl. No.: 15/525,803

(22) PCT Filed: Nov. 12, 2015
(86) PCT No.: PCT/GB2015/053432

§ 371 Date: May 10, 2017

(30) Foreign Application Priority Data
Nov. 13, 2014 (GB) 1420218.8

(51) Int. Cl. C12Q 1/6897; G01N 33/50

(52) U.S. Cl. CPC C12Q 1/6897; G01N 33/5041

(57) ABSTRACT

A method for the identification of new therapeutic targets and protein interaction sites for use in drug discovery. In particular the invention relates to a method for identifying inhibitors of a cell signalling pathway, the method comprising (1) providing a population of mammalian cells, each mammalian cell having an active cell signalling pathway and comprising: (a) a first heterologous nucleic acid comprising; (i) a nucleotide sequence encoding a first detectable reporter and, (ii) a constitutive regulatory element which is operably linked to the nucleotide sequence; and, (b) a second heterologous nucleic acid comprising: (i) a first nucleotide sequence encoding a repressor molecule, for example an RNA or protein, which inactivates, inhibits or suppresses expression of the first detectable reporter, (ii) a second nucleotide sequence encoding a second detectable reporter; and (iii) a signal-activated regulatory element which is activated by said cell signalling pathway, said signal-activated regulatory element being operably linked to the first and second nucleotide sequences, (2) introducing a library of test compounds into said population of mammalian cells, and; (3) determining the expression of the first and the second detectable reporters in one or more of the population of transfected cells, wherein expression of the first detectable reporter but not the second detectable reporter in a transfected cell is indicative that the test biomolecule expressed by the nucleic acid in the cell inhibits said cell signalling pathway.

### FIELD OF THE INVENTION

This invention relates to methods for the identification of new therapeutic targets and protein interaction sites for use in drug discovery.

### BACKGROUND OF THE INVENTION

The identification of new therapeutic targets is a key starting point for drug discovery. Drug discovery efforts have traditionally been focussed upon identifying classically-druggable targets such as kinases, G-protein coupled receptors (GPCRs) and ion channels. However, the ability to screen new classes of targets would allow the expansion of the ‘druggable genome’ and the identification of new therapeutic targets. Targets involved in protein:protein interactions (PPIs) are of particular interest, particularly PPIs which are involved in the signalling pathways which are utilised by cancer cells.

Functional screening and validation of candidate drug targets that are linked to disease biology has been performed typically at the genomic level, for example using gene knock-outs and the transcriptomic level (RNAi), where the number of targets is relatively limited (˜25,000 genes, with a subset of these having a number of alternative splicing forms). This level of complexity has not placed serious technical restrictions on assaying each gene-form sequentially for its function and essentiality in any disease setting of interest.

However, complexity at the proteomic level is substantially higher, with each protein adopting many different conformations during their normal or disease-context functions, which can offer numerous opportunities to therapeutically alter their activity. Because of this complexity, there have been few efforts to globally identify and functionally validate targets at the protein level, and the drug-binding sites therein. Although the concept has been proven using peptide libraries (<10,000 library size) in a ‘well-by-well’ basis to probe target function in phenotypic screening formats, this low-throughput approach is insufficient to routinely isolate new drug target sites for therapeutic intervention.

The current invention provides a method that allows the full complexity of the proteome (or genome) to be screened in pooled phenotypic assay formats, with a high degree of accuracy, enabling a clear and routine linkage between target and disease to be established. The invention further provides for the identification of protein interaction sites associated with disease.

### SUMMARY OF THE INVENTION

This invention relates to the development of screening methods for peptides and nucleic acids which inhibit or block cell signalling pathways. These methods may be useful, for example, in identifying and characterising target proteins and also protein:protein interaction (PPI) sites for use as drug targets for the modulation of these pathways.

One aspect of the invention provides a method of screening for a compound which inhibits a cell signalling pathway comprising;

(1) providing a mammalian cell having an active cell signalling pathway, wherein the cell comprises:
(a) a first heterologous nucleic acid comprising;
   (i) a nucleotide sequence encoding a first detectable reporter and,
   (ii) a constitutive regulatory element which is operably linked to the nucleotide sequence; and,
(b) a second heterologous nucleic acid comprising:
   (i) a first nucleotide sequence encoding a repressor molecule, for example an RNA or protein, which inactivates, inhibits or suppresses expression of the first detectable reporter,
   (ii) a second nucleotide sequence encoding a second detectable reporter; and,
   (iii) a signal-activated regulatory element which is activated by said cell signalling pathway,
      said signal-activated regulatory element being operably linked to the first and second nucleotide sequences,
      (2) introducing a test compound to the cell and;
      (3) determining the expression of the first and the second detectable reporters in the cell,
      wherein the presence of expression of the first detectable reporter and the absence of expression of the second detectable reporter in the mammalian cell is indicative that the test compound inhibits the cell signalling pathway.

The test compound may be a biomolecule which is introduced to the cell by contacting the cells with the biomolecule or by expressing a nucleic acid encoding a test biomolecule in the cell.

A method may comprise identifying a mammalian cell which expresses the first detectable reporter but not the second detectable reporter.

Another aspect of the invention provides a method of screening for a compound which inhibits a cell signalling pathway comprising:

(1) providing a population of mammalian cells, each mammalian cell having an active cell signalling pathway and comprising:
(a) a first heterologous nucleic acid comprising;
   (i) a nucleotide sequence encoding a first detectable reporter and,
   (ii) a constitutive regulatory element which is operably linked to the nucleotide sequence; and,
(b) a second heterologous nucleic acid comprising:
   (i) a first nucleotide sequence encoding a repressor molecule, for example an RNA or protein, which inactivates, inhibits or suppresses expression of the first detectable reporter,
   (ii) a second nucleotide sequence encoding a second detectable reporter; and,
   (iii) a signal-activated regulatory element which is activated by said cell signalling pathway,
      said signal-activated regulatory element being operably linked to the first and second nucleotide sequences,
      (2) introducing a library of test compounds into said population of mammalian cells, and;
      (3) determining the expression of the first and the second detectable reporters in one or more of the population of transfected cells,
      wherein expression of the first detectable reporter but not the second detectable reporter in a transfected cell is indicative that the test biomolecule expressed by the nucleic acid in the cell inhibits said cell signalling pathway.

A library of test compounds may be introduced into a population of mammalian cells by expressing a library of nucleic acids encoding a diverse population of test biomolecules in said population of mammalian cells.

A method may further comprise;

(4) identifying one or more cells in the population which express the first detectable reporter but not the second detectable reporter, said cells containing a nucleic acid encoding a putative inhibitor of a cell signalling pathway.

Another aspect of the invention provides a method of identifying a protein, protein region or protein:protein interaction (PPI) site, which may be a useful target, for example, for the therapeutic modulation, e.g. inhibition, of a cell signalling pathway or the development of therapeutics for the modulation, e.g. inhibition, of a cell signalling pathway, the method comprising:

(1) providing a population of mammalian cells having an active cell signalling pathway and comprising:
(a) a first heterologous nucleic acid comprising;
   (i) a nucleotide sequence encoding a first detectable reporter and,
   (ii) a constitutive regulatory element which is operably linked to the nucleotide sequence; and,
(b) a second heterologous nucleic acid comprising:
   (i) a first nucleotide sequence encoding a repressor molecule, for example an RNA or protein, which inactivates, inhibits or suppresses expression of the first detectable reporter,
   (ii) a second nucleotide sequence encoding a second detectable reporter; and,
   (iii) a signal-activated regulatory element which is activated by said cell signalling pathway,
      said signal-activated regulatory element being operably linked to the first and second nucleotide sequences,
      (2) introducing a library of test compounds into said population of mammalian cells, and;
      (3) determining the expression of the first and the second detectable reporters in the transfected cells,
      wherein the expression of the first detectable reporter but not the second detectable reporter in one or more transfected cells is indicative that the test biomolecule expressed in said one or more transfected cells is an inhibitor of said cell signalling pathway, and
      (4) identifying one or more transfected cells in the population which express the first detectable reporter but not the second detectable reporter.

One or more transfected cells which express the first detectable reporter but not the second detectable reporter may be isolated.

The nucleic acid encoding the test biomolecule from said one or more transfected cells may be amplified, cloned and/or sequenced.

The nucleic acid encoding the test biomolecule may be expressed to produce the test biomolecule.

An intracellular binding partner which binds the test biomolecule may be identified, said binding partner being a candidate target protein for modulation of a cell signalling pathway.

The region of the intracellular binding partner which binds to the test biomolecule may be identified, said region being a candidate target region or site for modulation of a cell signalling pathway.

The target protein, protein region or protein:protein interaction (PPI) site may modulate a phenotypic response in a mammalian cell.

Another aspect of the invention provides a mammalian cell or a population of mammalian cells, each cell comprising:
(a) a first heterologous nucleic acid comprising;
   (i) a nucleotide sequence encoding a first detectable reporter and,
   (ii) a constitutive regulatory element which is operably linked to the nucleotide sequence; and,
(b) a second heterologous nucleic acid comprising:
   (i) a first nucleotide sequence encoding a repressor molecule, for example an RNA or protein, which inactivates, inhibits or suppresses expression of the first detectable reporter,
   (ii) a second nucleotide sequence encoding a second detectable reporter; and,
   (iii) a signal-activated regulatory element which is activated by said cell signalling pathway,
      said signal-activated regulatory element being operably linked to the first and second nucleotide sequences.

In some embodiments, the cell or population may be transfected with a nucleic acid encoding a test biomolecule or a library of nucleic acids encoding a diverse population of test biomolecules, respectively.

The advantage provided by the invention described herein is that it allows the full complexity of the proteome to be screened for its function in a live cell ‘phenotypic’ assay format with peptide libraries (or RNAi, or genome editing libraries) comprising 10⁹ sequence diversity. This method permits strong positive selection and the isolation of a small number of true hit peptides (or nucleic acids) from a very large number of non-hit peptide (or nucleic acid) sequences enabling a clear linkage with disease to be established. The advantage of screening peptide libraries is that they, like small molecule drugs, typically act by directly and acutely inhibiting target function (rather than eliminating the target's long-term expression), the key to this method is turning a ‘negative’ cell phenotype signal (e.g., shut down of a signalling pathway or disruption of a protein/protein interaction) into a positive signal that can be selected for, or isolated from a large pool of cells harbouring inactive peptide sequences.

The identification of targets and their druggable sites that can be directly linked to disease, in a precise, efficient and reliable manner offers clear advantages in the development of suitable drug candidates for the treatment of disease. In particular, the method of the invention provides for the identification of intracellular targets and key druggable sites that play a role in disease progression, that may not otherwise be identifiable in other assay formats such as gene knock-out and RNA knock-down studies.

Another aspect of the invention provides a vector or combination of vectors which comprises:
(a) a first heterologous nucleic acid comprising;
   (i) a nucleotide sequence encoding a first detectable reporter and,
   (ii) a constitutive regulatory element which is operably linked to the nucleotide sequence; and,
(b) a second heterologous nucleic acid comprising:
   (i) a first nucleotide sequence encoding a repressor molecule, for example an RNA or protein, which inactivates, inhibits or suppresses expression of the first detectable reporter,
   (ii) a second nucleotide sequence encoding a second detectable reporter; and,
   (iii) a signal-activated regulatory element which is activated by said cell signalling pathway,
      said signal-activated regulatory element being operably linked to the first and second nucleotide sequences.

### BRIEF DESCRIPTION OF THE DRAWINGS

FIG. 1 shows a schematic representation of a screening method according to an embodiment of the invention. FIG. 1A shows that activation of the signalling pathway leads to activation of the transcriptional response element, which drives expression of EmGFP and the miRNA which then blocks the expression of the Cherry reporter. In this scenario, a cell would fluoresce green. FIG. 1B shows that, when the signalling pathway is activated, but is blocked by an inhibitor, the transcriptional response element is not activated. In this case, neither emGFP nor the miRNA which blocks Cherry are activated. This allows the expression of Cherry and the cell fluoresces red.

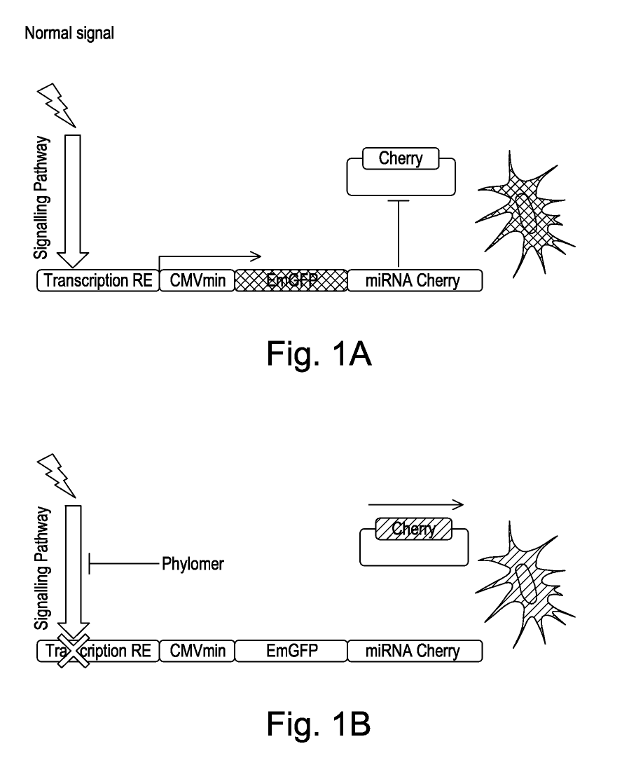

FIG. 2 shows a summary of the FACS phenotypes generated by screening methods according to some embodiments of the invention.

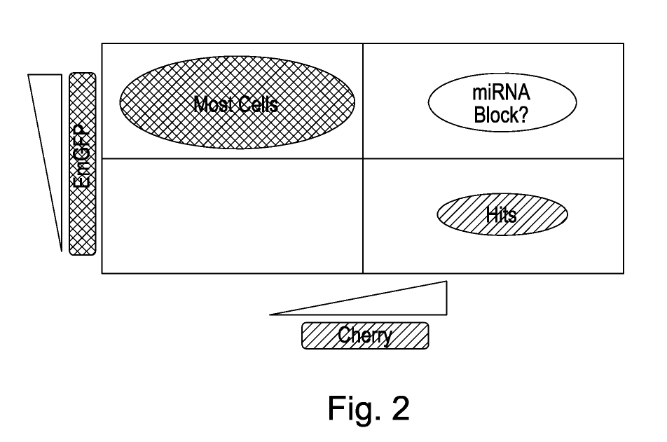

FIG. 3 shows different vectors tested in HeLa cells. Cherry constructs transiently transfected in HeLa cells, scanned live on the Arrayscan 01Sep11. Intensities can be directly compared.

FIG. 4 shows different vectors tested in C3H10T1/2 cells.

FIG. 5 shows the effect of ARE on mCherry plasmid stability in transiently transfected U2OS cells.

### DETAILED DESCRIPTION OF INVENTION

This invention relates to cell-based screening methods for the identification of biomolecules which inhibit cell-signalling pathways and the identification of proteins and surface sites of PPIs on proteins which participate in signal transduction and may be useful as drug targets to modulate cell-signalling pathways, in particular pathways which are active in cancer cells.

A cell signalling pathway is a series of interacting factors in a cell which transmits an intracellular signal within the cell in response to an extracellular stimulus at the cell surface and leading to changes in cell phenotype. Transmission of signals along a cell signalling pathway results in the activation of one or more transcription factors which alter gene expression. Preferred cell signalling pathways display aberrant activity, for example activation, up-regulation or mis-regulation in diseased cells, such a cancer cells. For example a pathway may be constitutively activated (i.e. permanently switched on) in a cancer cell, or inappropriately activated by an extracellular ligand, for example in an inflammatory cell in rheumatoid arthritis.

A functional cell signalling pathway is a pathway which is intact and capable of transmitting signals, if the pathway is switched on or activated, for example by an appropriate extracellular stimulus. An active cell signalling pathway is a pathway which has been switched on, for example by an appropriate extracellular stimulus and is actively transmitting signals.

Suitable cell signalling pathways include any signalling pathway which results in a transcriptional event in response to a signal received by a cell which results in a transcriptional event.

Cell signalling pathways for investigation as described herein may include cell signalling pathways which may be activated in cancer cells, such as Ras/Raf, Hedgehog, Fas, Wnt, Akt, ERK, TGFβ, and Notch signalling pathways.

Mammalian cells for use in the methods described herein may be any cultured mammalian cell, for example a human or non-human cell, in which the cell signalling pathway is functional.

During the screening methods described herein, the cell signalling pathway of interest is active in the mammalian cell or mammalian cell population. In the cell or cells, the active pathway activates the signal-activated regulatory element which causes expression of the second detectable reporter and the repressor molecule. The repressor molecule then suppresses expression of the first detectable reporter, such that the cells express the second detectable reporter, but not the first.

The cell signalling pathway of interest may be activated by any suitable technique.

In some embodiments, the cell signalling pathway of interest may be constitutively activated in the mammalian cell i.e. the signalling pathway is permanently switched on and active in the cell. For example, a mammalian cell may have a mutation, preferably in an upstream pathway component of the pathway, such as a cell surface receptor, which causes constitutive activation of the pathway. For example, Hedgehog signalling is constitutively active in osteosarcoma cell lines (Hirotsu et al. Molecular Cancer 2010, 9:5 http://www.molecular-cancer.com/content/9/1/5).

In other embodiments, the cell signalling pathway of interest may be activated in the mammalian cell by transfection of a mutant protein which causes constitutive activation. The activation of cell signalling pathways in cell lines through mutations is well known in the art. For example, Smoothened M2 mutants (SmoM2) are known to cause activation of signalling pathways in human cancer cells.

In other embodiments, the cell signalling pathway may be activated in the mammalian cell by an appropriate extracellular stimulus. For example, the cell may be treated with a ligand which activates the pathway by binding to a cell surface receptor. The ligand may be a drug or a natural ligand. For example, a cell may be treated with recombinant sonic hedgehog protein to activate hedgehog signalling.

Mammalian cells for use in the methods described herein comprise at least two heterologous nucleic acids (i.e. first and second heterologous nucleic acids).

A heterologous nucleic acid is a nucleic acid molecule which does not exist naturally in the mammalian cell. Heterologous nucleic acids may be recombinant or synthetic and may be introduced into a mammalian cell by any suitable molecular biology technique, such as transformation or transfection. A heterologous nucleic acid may be extra-chromosomal or may be incorporated into the genome of a mammalian cell.

A mammalian cell may comprise a first heterologous nucleic acid which comprises a first reporter coding sequence encoding a first detectable reporter.

A detectable reporter is a polypeptide which can be detected when it is expressed in the cell may be detected. For example, expression of the detectable reporter may lead to the production of a signal, for example a fluorescent, bioluminescent or colorimetric signal, which can be detected using routine techniques. The signal may be produced directly from the reporter, after expression, or indirectly through a secondary molecule, such as a labelled antibody.

Suitable detectable reporters include fluorescent proteins which produce a detectable fluorescent signal. Suitable fluorescent reporters are well known in the art and include Y66H, Y66F, EBFP, EBFP2, Azurite, GFPuv, T-Sapphire, TagBFP, Cerulean, mCFP, ECFP, CyPet, Y66W, dKeima-Red, mKeima-Red, TagCFP, AmCyanl, mTFP1 (Teal), S65A, Midoriishi-Cyan, Wild Type GFP, S65C, TurboGFP, TagGFP, TagGFP2, AcGFP1, S65L, Emerald, S65T, EGFP, Azami-Green, ZsGreen1, Dronpa-Green, TagYFP, EYFP, Topaz, Venus, mCitrine, YPet, TurboYFP, PhiYFP, PhiYFP-m, ZsYellow1, mBanana, Kusabira-Orange, mOrange, mOrange2, mKO, TurboRFP, tdTomato, DsRed-Express2, TagRFP, DsRed monomer, DsRed2 (“RFP”), mStrawberry, TurboFP602, AsRed2, mRFP1, J-Red, mCherry, HcRed1, mKate2, Katushka (TurboFP635), mKate (TagFP635), TurboFP635, mPlum, mRaspberry, mNeptune and E2-Crimson.

Suitable fluorescent reporters are available commercially (Clontech Labs Inc USA, Evrogen Moscow, RU; MBL Int MA USA; Addgene Inc MA USA).

Suitable detectable reporters also include cell surface markers which contain epitopes not otherwise present on the cell surface (i.e. unique epitopes). Expression of a cell surface marker may be detected using a labelled antibody which binds to the marker and produces a detectable signal.

Any cell surface marker which is not normally expressed on the mammalian cell may be used. For example, immune cell markers such as CD8 and CD19 may be employed, when the mammalian cells are not non-immune cells which do not express CD8 and CD19.

Other reporters include gene products (such as enzymes) whose expression in a mammalian cell can be detected with a live cell assay (i.e. an assay which does not require cell fixation or lysis). For example, a suitable reporter might include β-galactosidase, which can be detected using substrates such as CMFDG which fluoresces upon β-galactosidase-dependent catalysis.

The detectable reporter may comprise an in-frame destabilization sequence, for example a PEST sequence, within the coding sequence of the reporter. A PEST sequence is a peptide sequence that is rich in proline (P), glutamic acid (E), serine (S), and threonine (T).

The detectable reporter may further comprise a CL1 degron sequence at the C-terminal. CL1 degron sequence down regulates expression by specifically targets proteins for proteosomal degradation.

The detectable reporter may comprise an in-frame destabilization sequence, for example a PEST sequence and/or a CL1 degron sequence at the C-terminal.

The nucleotide sequence which encodes the first detectable reporter is operably linked to a constitutive regulatory element.

A regulatory element is a sequence of nucleotides from which transcription may be initiated of a nucleotide sequence operably linked downstream (i.e. in the 3′ direction on the sense strand of double-stranded DNA).

A nucleotide sequence which is operably linked to a regulatory element is joined as part of the same nucleic acid molecule, suitably positioned and oriented for transcription to be initiated from the regulatory element.

A constitutive regulatory element causes the nucleotide sequence to be transcribed at a constant rate in the mammalian cell independently of the presence or absence of extracellular stimuli. Suitable constitutive regulatory elements for expression in mammalian cells are well-known in the art and include viral promoters, such as CMV, SV40, HSV-TK, UBC and EF-1α.

In particular embodiments of the invention, the constitutive regulatory element is selected from the CMV promoter and HSV-TK.

The nucleotide sequence encoding the first detectable reporter is transcribed at a constant rate in the mammalian cells. The first detectable reporter is therefore expressed in the mammalian cell at a constant level or substantially constant level in the absence of repressor molecule. Expression of the repressor molecule abolishes expression of the first detectable reporter, despite a constant rate of transcription from the coding sequence.

A mammalian cell may comprise a second heterologous nucleic acid which comprises a first nucleotide sequence encoding a second detectable reporter.

Suitable detectable reporters are described above.

The second detectable reporter is different from the first detectable reporter i.e. the first and second detectable reporters directly or indirectly produce signals which can be distinguished from each other. For example, the first and the second detectable reporters may be first and second fluorescent proteins which fluoresce at different wavelengths; first and second cell surface markers which bind to different antibodies or the first detectable reporter may be one of a fluorescent protein and a cell surface marker and the second detectable reporter may encode the other of a fluorescent protein and a cell surface marker.

Suitable reporter sequences and combinations of reporter sequences are well known in the art.

In some preferred embodiments, the first and second detectable reporters are fluorescent proteins which fluoresce at different wavelengths. Suitable fluorescent proteins are described above. The first and second detectable reporters may be any pair of fluorescent proteins whose emission wavelengths are sufficiently different to allow resolution, for example by flow cytometry. Suitable pairs of fluorescent proteins include Cherry and emGFP.

In other embodiments, the first and second detectable reporters are cell surface markers which bind to first and second labelled antibodies, for example, fluorescently-labelled antibodies. The first and second antibodies may have different labels to allow the expression of the first and second detectable reporters to be distinguished.

The second heterologous nucleic acid may comprise a second nucleotide sequence which encodes a repressor which inactivates, inhibits or suppresses expression of the first detectable reporter.

Suitable repressor molecules include peptides and polypeptides such as protein aptamers, such as phylomers and antibody molecules, such as domain antibodies, nanobodies or scFv, which inhibit or inactivate the first detectable reporter.

Suitable repressor molecules include RNA molecules, such as miRNA, RNAi, siRNA, shRNA, ribozyme or antisense RNA, which suppress the expression of the first detectable reporter.

For example, expression of the first detectable reporter may be inhibited using anti-sense or sense technology. The use of these approaches to down-regulate gene expression is now well-established in the art.

In one example the repressor molecule is miRNA e.g. miRNA Cherry.

Anti-sense oligonucleotides may be designed to hybridise to the complementary sequence of nucleic acid, pre-mRNA or mature mRNA, interfering with the production of the first detectable reporter so that its expression is completely or substantially completely prevented. In addition to targeting coding sequence of the first detectable reporter, anti-sense techniques may be used to target control sequences of the first detectable reporter, e.g. in the 5′ flanking sequence, whereby the anti-sense oligonucleotides can interfere with expression control sequences. The construction of anti-sense sequences and their use is described for example in Peyman and Ulman, Chemical Reviews, 90:543-584, (1990) and Crooke, Ann. Rev. Pharmacol. Toxicol. 32:329-376, (1992).

Transcription of the anti-sense strand of the second nucleotide sequence yields RNA which is complementary to normal mRNA transcribed from the sense strand of the first detectable reporter. The complementary anti-sense RNA sequence binds with first detectable reporter mRNA to form a duplex, inhibiting translation of the first detectable reporter mRNA from the first detectable reporter into protein.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the first detectable reporter sequence inserted in sense, that is the same, orientation as the target gene, to achieve reduction in expression of the first detectable reporter by co-suppression; Angell & Baulcombe (1997) The EMBO Journal 16, 12:3675-3684; and Voinnet & Baulcombe (1997) Nature 389: pg 553). Double stranded RNA (dsRNA) has been found to be even more effective in gene silencing than both sense and antisense strands alone (Fire A. et al Nature 391, (1998)). dsRNA mediated silencing is gene specific and is often termed RNA interference (RNAi).

RNA interference is a two-step process. First, dsRNA is cleaved within the cell to yield short interfering RNAs (siRNAs) of about 21-23nt length with 5′ terminal phosphate and 3′ short overhangs (˜2nt). The siRNAs target the corresponding mRNA sequence specifically for destruction (Zamore P. D. Nature Structural Biology, 8, 9, 746-750, 15 (2001)).

The use of miRNA, RNAi, siRNA and shRNA molecules to suppress gene expression is well known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Another possibility is that transcription of the second nucleotide sequence on produces a ribozyme, able to cut nucleic acid at a specific site—thus also useful in influencing expression of the first detectable reporter. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

An RNA molecule such as a siRNA, dsRNA or miRNA may comprise a partial sequence of the first detectable reporter mRNA, for example at least 10 nucleotides, at least 15 or at least 20 nucleotides of the first detectable reporter sequence.

Suitable RNA molecules for down-regulation of the first detectable reporter may possible 85% or more, 90% or more, 95% or more or 100% sequence identity with a contiguous sequence of 10 to 40 nucleotides from the first detectable reporter mRNA sequence.

In some preferred embodiments, the second nucleotide sequence encodes a miRNA which suppresses expression of the first detectable reporter.

A miRNA is a short RNA molecule of typically 21-25 nucleotides which specifically hybridises to the first detectable reporter mRNA in a mammalian cell and inhibits the translation or degradation of the mRNA and thus the expression of the encoded protein (Brown, B D and Naldini, L. Nat. Rev. Genetics; 2009; 10; 578-585). miRNA may therefore be used to specifically suppress the expression of the first detectable reporter. Suitable miRNA molecules for the suppression of any specific detectable reporter may be designed and produced using techniques which are well-known in the art (e.g. S. Ossowski, R et al Plant J. 53 (2008) 674-690; John et al, PLoS Biology, 11(2), 1862-1879, 2004). Various web-based tools are available to design primers to any specific target gene, including detectable reporter genes (e.g. WMD3 Web MicroRNA Designer Ossowski et al Max Planck Institute for Developmental Biology, Tubingen http://wmd3.weigelworld.org/cgi-bin/webapp.cgi?page=Help).

For example, a miRNA for the suppression of Cherry expression may have the nucleotide sequence according to SEQ ID No: 1

The mRNA of the reporter protein may comprise AU-Rich Elements (Adenylate-uridylate-rich elements or AREs) into the 3′ UTR (3′ untranslated region) of its mRNA. AREs are regions that contain repeated adenine and uridine bases and are a key factor in determining the stability of mRNA in mammalian cells (Chen, Chyi-Ying A et al, 1995, Trends in Biochemical Science, 20 (11): 465-470).

The sequences encoding the second detectable reporter and the miRNA are operably linked to a signal-activated regulatory element. In other words, the same signal-activated regulatory element initiates transcription of both the coding sequences (i.e. the sequences are co-cistronic).

A signal-activated regulatory element is a regulatory element which initiates transcription of operably linked coding sequences when a cell signalling pathway of interest is actively signalling (i.e. the pathway is active) and does not initiate transcription of operably linked coding sequences when the cell signalling pathway is not actively signalling (i.e. the pathway is inactive). A pathway may be inactive if it is not switched on, for example due to the absence of an appropriate extracellular stimulus, or if one or more steps or components of the pathway are blocked or inhibited.

For example, active signalling through a cell signalling pathway may lead to the activation of a transcription factor in the mammalian cell. Once activated, the transcription factor may then bind to one or more specific binding sites in the signal-activated regulatory element. This binding activates the regulatory element and switches on the transcription of the operably linked coding sequences.

The choice of signal-activated regulatory element will depend on the cell signalling pathway and the transcription factors which is activated by the pathway. For example, the Notch signalling pathway activates the transcription factor RBP-Jkappa (aka CBF-1) which binds to the nucleotide sequence GTGGGAA. A Notch signal-activated regulatory element may therefore comprise one or more copies of this sequence. The Wnt signalling pathway activates the transcription factor LEF1 which binds to the nucleotide sequence AGATCAAAGG. A Wnt signal-activated regulatory element may therefore comprise one or more copies of this sequence.

A signal-activated regulatory element may comprise one or more transcription factor binding sites linked to a minimal promoter sequence, such as a CMV minimal promoter.

Suitable signal-activated regulatory elements may be selected from the CMV promoter and the HSV-TK promoter, for example, to drive expression.

Nucleic acid as described herein may be readily prepared by the skilled person using standard techniques (for example, see Molecular Cloning: a Laboratory Manual: 3rd edition, Sambrook and Russell (2001) Cold Spring Harbor Laboratory Press; Molecular Biology, Second Edition, Ausubel et al. eds. John Wiley & Sons, 1992; Recombinant Gene Expression Protocols Ed RS Tuan (March 1997) Humana Press Inc). For example, first and second heterologous nucleic acids may be prepared by conventional solid phase synthesis techniques or may be produced by recombinant means.

The first and second heterologous nucleic acids may be comprised within the same or separate vectors. Separate vectors may be the same or different. Suitable vectors can be chosen or constructed, containing appropriate regulatory sequences, including promoter sequences for driving transcription of the coding nucleotide sequence, terminator fragments, polyadenylation sequences, enhancer sequences, marker genes and other sequences as appropriate, for expression in mammalian cells as described herein. Suitable vectors for expressing nucleic acid in mammalian cells are well known. For example, vectors may be plasmids, or viral vectors, such as adenovirus, adeno-associated virus, retrovirus (such as HIV, MLV and pMX-based vectors), lentivirus or alpha-virus vectors.

When the cell signalling pathway is active, the signal-activated regulatory element in the second heterologous nucleic acid in the mammalian cell is switched on and initiates transcription of the nucleotide sequences encoding the second detectable reporter and the repressor molecule. The mammalian cell therefore expresses the second detectable reporter and the repressor molecule, when the cell signalling pathway is active. Expression of the repressor molecule prevents expression of the first detectable reporter in the cell. The mammalian cell does not therefore express the first detectable reporter when the cell signalling pathway is active. In other words, when the cell signalling pathway is active, the mammalian cells express the second but not the first detectable reporter.

When the cell signalling pathway is inactive, for example when one or more steps or components are blocked or inhibited, the signal-activated regulatory element in the second heterologous nucleic acid in the mammalian cell is not switched on and there is no transcription of the nucleotide sequences encoding the second detectable reporter and the repressor molecule. The mammalian cell does not therefore express either the second detectable reporter or the repressor molecule, when the cell signalling pathway is inactive. The absence of repressor molecule expression means there is no suppression or inhibition of the first detectable reporter in the cell. The mammalian cell therefore expresses the first detectable reporter, when the cell signalling pathway is inactive. In other words, when the cell signalling pathway is inactive, the mammalian cells express the first but not the second detectable reporter.

When appropriately stimulated or constitutively activated, the cell signalling pathway may be inactive in the presence of a compound, such as a biomolecule, which inhibits a component of the pathway or blocks or inhibits a step in the pathway, for example by inhibiting a protein:protein interaction.

Detection of the expression of the first and second detectable reporters in the mammalian cell (i.e. the reporter phenotype) is therefore indicative of the inhibition of the cell signalling pathway. A mammalian cell which is negative for the first detectable reporter and positive for the second detectable reporter has an active signalling pathway and a cell which is positive for the first detectable reporter and negative for the second detectable reporter has an inactive (e.g. a blocked or inhibited) signalling pathway.

When a test compound, such as a biomolecule, is introduced to the mammalian cell under conditions in which the cell signalling pathway is normally active, the expression of the first detectable reporter without expression of the second detectable reporter is indicative that the compound is an inhibitor of the cell signalling pathway.

Suitable test molecules include small organic molecules (i.e. non-polymeric organic molecules with a molecular weight of less than 800 Da) or biomolecules, such as peptide and nucleic acid aptamers, antibody molecules, such as domain antibodies, nanobodies or scFv, and suppression RNA, such as RNAi, siRNA, shRNA, ribozyme or antisense RNA.

Test biomolecules encoded by a heterologous nucleotide sequence may be used in the methods described herein.

Preferably, the test biomolecule is a peptide aptamer.

A peptide aptamer is a short peptide sequence, for example 15 to 80 amino acids which binds specifically to a site on a target protein. In some embodiments, a peptide aptamer is contained within a stable scaffold protein, for example, a non-immunoglobulin scaffold such as fibronectin (Adnectin™), ankyrin (DARPin™, lipocalin (Anticalin™), trinectin, a kunitz domain, transferrin, nurse shark antigen receptor or sea lamprey leucine-rich repeat protein (Binz et al Nat Biotech 23 1257-1268 (2005)).

The sequence of a peptide aptamer may be fully or partially random or may be non-random.

In preferred embodiments, the peptide aptamer is a phylomer. A phylomer is a peptide of 15 to 80 amino acids, preferably 15 to 50 amino acids, encoded by a short fragment of nucleotide sequence, for example 45 to 240 nucleotides, from a microbial nucleic acid.

Microbial nucleic acid used to generate phylomers may include genomic DNA, RNA or cDNA obtained from one or more different micro-organisms, such as bacteria, Archaea or lower eukaryotes. Phylomers may be encoded by any reading frame of a fragment of nucleotide sequence. Preferably, the phylomer is encoded by a natural open reading frame (ORF) of the nucleotide sequence.

Phylomers and phylomer libraries are known in the art (Watt et al (2006) Nat Biotech 24 17-183; Watt et al (2006) Expert Opin Drug Disc 1 491-502, Watt et al (2009) Future Med Chem 1 (2) 257-265, WO2005/119244; WO/2004/074479, and, WO/2006/017913).

A phylomer library is a population of phylomers having diverse sequences. For example, a phylomer library may comprise 3×10⁴ or more, 1×10⁵ or more, 1×10⁶ or more, 1×10⁷ or more, 1×10⁸ or more different phylomer sequences, preferably 1×10⁸ to 1×10⁹.

Phylomer libraries may be constructed using any convenient technique. For example, a phylomer library may be constructed by randomly cloning short fragments of nucleotide sequence from one or more microbial nucleic acids into expression vectors. A phylomer library may be produced by a method comprising;
(i) producing fragments from nucleic acids from two or more microorganisms;
(ii) inserting the nucleic acid fragments into an expression vector adapted to express the fragment; and
(iii) expressing the peptide encoded by the nucleic acid fragment.

The nucleic acid fragments may be produced from genomic DNA, cDNA, or amplified nucleic acid from one or more microbial genomes or transcriptomes, preferably genomes.

The nucleic acid fragments may be produced from a mixture of nucleic acids (i.e. genomes or transcriptomes) from different microorganisms. The nucleic acids may be present in the mixture in an amount that is proportional to the complexity and size of the genome (or transcriptome), for example, in comparison to the complexity and size of other genomes in the mixture. This results in approximately equal representation of the genome fragments.

Preferably, a library of phylomers is produced from a mixture of two or more phylogenetically diverse microbial genomes or transcriptomes, including for example, genomic DNA of cDNA from extremophiles, for example thermophilic Archea and bacteria, such as Archaeoglobus fulgidus, Aquifex aeolicus, Aeropyrum pernix, Pyrococcus horikoshii, Synechocystis PCC 6803, Thermoplasma volcanium, Thermotoga maritima, Thermus thermophilus, Methanobacterium thermoautotrophicum, Methanococcus jannaschii and Deinococcus radiodurans.

Examples of phylomer libraries are described in EP1696038.

Nucleic acid fragments may be generated from one, two or more microbial genomes or transcriptomes by one or more of a variety of methods known to those skilled in the art. Suitable methods include, for example, mechanical shearing (e.g. by sonication or passing the nucleic acid through a fine gauge needle), digestion with a nuclease (e.g. Dnase 1), digestion with one or more restriction enzymes, preferably frequent cutting enzymes that recognize 4-base restriction enzyme sites and treating the DNA samples with radiation (e.g. gamma radiation or ultra-violet radiation). In some embodiments, nucleic acid fragments may be generated from one, two or more microbial genomes or transcriptomes by polymerase chain reaction (PCR) using, for example, random or degenerate oligonucleotides. Random or degenerate oligonucleotides may include restriction enzyme recognition sequences to allow for cloning of the amplified nucleic acid into an appropriate nucleic acid vector.

Each fragment of microbial nucleic acid produced as described above encodes a phylomer. The fragments may be cloned into expression vectors for expression of the phylomer.

Nucleic acid encoding a test biomolecule may be flanked (for example 5′ and 3′ to the coding sequence) by specific sequence tags. Sequence tags comprise 10 to 50 nucleotides of known sequence which may be used as binding sites for oligonucleotide primers.

Preferably, the sequence of the tag is not found in the mammalian genome. This allows the coding sequence of a test biomolecule to be conveniently amplified from the mammalian cell, for example by PCR, if required.

The nucleic acid encoding the test biomolecule may be operably linked to a regulatory element and comprised in a vector for expression in the mammalian cell. Suitable regulatory elements and vectors are well-known in the art and described elsewhere herein. Suitable techniques for producing and manipulating nucleic acid and expressing it in mammalian cells are well-known in the art.

A method described herein may comprise transfecting a mammalian cell comprising first and second heterologous nucleic acids as described above with a nucleic acid encoding a test biomolecule to produce a transfected cell comprising a nucleic acid which encodes the test biomolecule.

In preferred embodiments, a population of mammalian cells is transfected with a library of nucleic acids encoding a diverse population of test biomolecules. A method described herein may comprise transfecting a population of mammalian cells comprising first and second heterologous nucleic acids as described above with a library of nucleic acids encoding a diverse population of test biomolecules to produce a population of transfected cells, each cell containing a nucleic acid which encodes a test biomolecule.

The library may be pooled to allow simultaneous transfection and screening of all the members of the library.

A suitable library of nucleic acids may, for example, encode 3×10⁴ or more, 1×10⁵ or more, 1×10⁶ or more, 1×10⁷ or more, 1×10⁸ or more different test biomolecules. A library may for example encode 10⁸ to 10⁹ different test biomolecules.

The nucleic acids in the library are preferably cloned into vectors for mammalian cell expression as described above.

After transfection of the mammalian cells and expression of the nucleic acids encoding 10 the test biomolecules, the population of cells may be screened for expression of the first and second detectable reporters as described above to identify cells in which the test biomolecule has inhibited the cell signalling pathway.

As described above, expression of the first detectable reporter in a mammalian cell in the population the absence of expression of the second reporter is indicative that the test compound introduced to the mammalian cell, for example, a test biomolecule expressed by the cell, is an inhibitor of the cell signalling pathway.

Suitable techniques for determining reporter expression will depend on the reporters which are used. Typically, the detectable reporters will be fluorescent proteins or markers detectable with fluorescently labelled antibodies. The fluorescence phenotype of the cells in the population may be determined by any convenient fluorescent techniques.

If one or both of the detectable reporters are cell surface markers, expression may be determined by contacting the population of cells with antibodies which bind to the reporters. The antibodies may be labelled, for example with a fluorescent label. If both detectable reporters are cell surface markers, the antibodies which bind to each marker may be labelled with fluorescent labels which are distinguishable. After labelling, the amount of label attached to each cell may be determined, for example by measuring fluorescence, to determine the expression of the reporter.

Mammalian cells with the desired phenotype of reporter expression may be isolated. For example, cells which express the first detectable reporter but not the second detectable reporter may be isolated from other cells in the population.

In some preferred embodiments, fluorescence activated cell sorting (FACS) may be used to detect reporter expression and isolate cells with a defined fluorescence phenotype, for example cells which are positive for the first detectable reporter and negative for the second detectable reporter. Suitable FACS techniques are well known in the art. (see for example Ormerod, M. G. (1999) Flow Cytometry. 2nd edition. BIOS Scientific Publishers, Oxford. ISBN 185996107X).

Any suitable FACS apparatus may be employed (e.g. MoFlo Astrios Cell Sorter (Beckman Coulter Inc, CA USA).

As described above, the test compound introduced to mammalian cells which are found to be positive for the first detectable reporter and negative for the second detectable reporter is a putative inhibitor of the cell signalling pathway. For example, a test biomolecule which is expressed in mammalian cells which are positive for the first detectable reporter and negative for the second detectable reporter is a putative inhibitor of the cell signalling pathway.

Nucleic acids encoding test biomolecules may be isolated from mammalian cells which are positive for the first detectable reporter and negative for the second detectable reporter.

Techniques for the isolation of nucleic acid from a mammalian cell are well-known in the art. For example, total DNA may be isolated from the cells and the nucleic acid encoding the test biomolecule may then be amplified from the isolated total DNA. In some preferred embodiments, the nucleic acid may be amplified using primers which hybridise to the sequence specific tags flanking the test biomolecule coding sequence.

Nucleic acids encoding test biomolecules or amplification products thereof may be cloned into vectors and/or sequenced.

Sequencing may be useful, for example, in identifying and distinguishing individual test biomolecule coding sequences from the population of mammalian cells positive for the first detectable reporter and negative for the second detectable reporter.

The population of test nucleic acids isolated from mammalian cells positive for the first detectable reporter and negative for the second detectable reporter in a first screen, or amplification products thereof, may be used in one or more further rounds of screening as described above to generate a sub-population which is enriched for sequences encoding inhibitory biomolecules. For example, a method may comprise;
(1) providing a population of mammalian cells comprising a first and a second heterologous nucleic acid, as described above;
(2) transfecting said population of cells with a population of test nucleic acids isolated from mammalian cells positive for the first detectable reporter and negative for the second detectable reporter in a first screen as described above, each cell containing a nucleic acid encoding a test biomolecule,
(3) expressing the population of nucleic acids in the population of transfected cells, and
(4) determining the expression of the first and the second detectable reporters in the transfected cells,
   wherein expression of the first detectable reporter but not the second detectable reporter is indicative that the test biomolecule expressed by a nucleic acid in a transfected cell is an inhibitor of said cell signalling pathway,
(5) identifying one or more transfected cells in the population which express the first detectable reporter but not the second detectable reporter, and
(6) isolating the one or more transfected cells which express the first detectable reporter but not the second detectable reporter.

One, two, three or more rounds of screening may be performed until one or more nucleic acids encoding inhibitory test biomolecules have been identified.

In some embodiments, the identified nucleic acids may be further manipulated, for example by re-cloning. In some embodiments, the nucleic acid may be cloned into an expression vector adjacent to nucleic acid encoding a heterologous peptide, such that the vector expresses a fusion protein comprising the biomolecule fused to the heterologous peptide. Suitable heterologous peptides include epitope tags, affinity tags and cell penetrating peptides (CPPs).

An epitope tag is a heterologous amino acid sequence which forms one member of a specific binding pair. Peptides containing an epitope tag may be isolated and/or detected through the binding of the other member of the specific binding pair to the epitope tag. For example, the epitope tag may be an epitope which is bound by an antibody molecule. Suitable epitope tags are well-known in the art including, for example, MRGS(H)₆, DYKDDDDK (FLAG™), T7-, S-(KETAAAKFERQHMDS), poly-Arg (R5-6), poly-His (H2-10), poly-Cys (C4) poly-Phe(F11) poly-Asp(D5-16), Strept-tag II (WSHPQFEK), c-myc (EQKLISEEDL), Influenza-HA tag (Murray, P. J. et al (1995) Anal Biochem 229, 170-9), Glu-Glu-Phe tag (Stammers, D. K. et al (1991) FEBS Lett 283, 298-302), Tag.100 (Qiagen; 12 aa tag derived from mammalian MAP kinase 2), Cruz tag 09™ (MKAEFRRQESDR, Santa Cruz Biotechnology Inc.) and Cruz tag 22™ (MRDALDRLDRLA, Santa Cruz Biotechnology Inc.). Other suitable tags include GST (glutathione-S-transferase), MBP (maltose binding protein), GAL4, β-galactosidase, biotin and strepavidin. Known tag sequences are reviewed in Terpe (2003) Appl. Microbiol. Biotechnol. 60 523-533.

Epitope tags may be useful in purifying and/or isolating the phylomer, for example for the immunoprecipitation of test biomolecules bound to cellular binding partners.

A CPP is a heterologous amino acid sequence which facilitates transport of an attached moiety across a cell membrane. Suitable CPPs are well-known in the art including, basic peptides, such as Drosophila homeoprotein antennapedia transcription protein (AntHD), HSV structural protein VP22, HIV TAT protein, Kaposi FGF signal sequence (kFGF), protein transduction domain-4 (PTD4), Penetratin, M918, Transportan-10, PEP-I peptide, nuclear localization sequences, amphipathic peptides, and peptide sequences comprising 5 or more contiguous basis residues, such as arginines or lysines (e.g. (R)₉, (K)₉, (R)₁₁, or (K)₁₁). Other suitable CPPs are known in the art (see for example Inoue et al., 2006 Eur. Urol. 49, 161-168; Michiue et al., 2005 J. Biol. Chem. 280, 8285-8289; Wadia and Dowdy, 2002 Curr. Opin. Biotechnol. 13 52-56; Langel (2002) Cell Penetrating Peptides, CRC Press, Pharmacology and Toxicology Series; U.S. Pat. No. 6,730,293, WO05/084158 and WO07/123667)). Wadia & Dowdy Current Opin Biotechnology (2002) 13 52-56; Wagstaff & Jans Curr Medicinal Chemistry 13 1371-1387 (2006).

CPPs may be useful in transporting a test biomolecule into a cell, for example to screen directly for effects on cell phenotype.

A test biomolecule which alters cellular phenotype, optionally fused to an epitope tag and/or a CPP, may be produced or synthesised.

Various approaches for the production of biomolecules are available. Encoding nucleic acid may be expressed to produce the test biomolecule (see for example, Recombinant Gene Expression Protocols Ed RS Tuan (March 1997) Humana Press Inc). Alternatively, test biomolecules may be generated wholly or partly by chemical synthesis. Test biomolecules may be synthesised using liquid or solid-phase synthesis methods; in solution; or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof. Chemical synthesis of peptides is well-known in the art (J. M. Stewart and J. D. Young, Solid Phase Peptide Synthesis, 2nd edition, Pierce Chemical Company, Rockford, Ill. (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A Users Manual, ABI Inc., Foster City, Calif.; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

Having identified a test biomolecule which alters cellular phenotype and produced the test biomolecule, optionally as a fusion protein, a method may further comprise confirming the effect of the test biomolecule on the phenotype of a mammalian cell. For example, test biomolecules which have been synthesised with a Cell-Penetrating Peptide (CPP) may be used directly on the cells in order to elicit a phenotypic deflection.

Inhibitory biomolecules expressed from test nucleic acids identified from a library as described above may be used to screen for intracellular binding partners, for example cellular proteins which bind to the biomolecule. For example, the expressed biomolecule may be used as a bait molecule to identify intracellular binding partners in a mammalian cell or cell extract. Cellular proteins which bind to the bait biomolecule may be isolated.

Suitable techniques for identifying intracellular binding partners are well known in the art. They include techniques such as radio immunoassay, co-immunoprecipitation, scintillation proximity assay and ELISA methods. For example, the biomolecule may be over-expressed in mammalian cells, immunoprecipitated with antibodies binding to the epitope tag. Proteins bound to the biomolecule may be analysed, for example by MALDI-linked TOF mass spectrometry, and identified.

A method may comprise identifying a cellular binding partner of a test biomolecule identified in a screen described above. For example, the cellular binding partner may be a protein which specifically interacts with or binds to the test biomolecule.

Since the test biomolecule inhibits a cellular signalling pathway, the cellular binding partner may be identified as a putative component of the pathway. This may be useful as a target for the development of therapeutics which modulate the pathway.

Following identification of the cellular binding partner, the binding site, region or domain of the cellular binding partner which interacts with the test biomolecule may be identified. This site region or domain may also be useful as a target site for the development of therapeutics which modulate the pathway.

For example, X ray crystallography, NMR or standard biochemical techniques, such as immunoprecipitation, based on series of deletion constructs may be performed. For example, test biomolecules may be co-crystallised with the target protein and the structure solved.

Following identification of a target protein by a method described herein, the interaction site of the target protein may be investigated.

The interaction site is the site or region at which the bait biomolecule binds to block the activity of the target protein. Since binding at the interaction site blocks activity, the interaction site is the site or region of a target protein through which the target protein binds to a binding partner. For example, the interaction site may be the site of a protein:protein interface when the target protein is bound to its binding partner.

Blockade of the interaction site e.g. by a small organic molecule, antibody or other biomolecule which binds at the site may disrupt binding of the target protein to a binding partner. Binding at the interaction site may therefore modulate the activity of the target protein and alter one or more phenotypic traits or characteristics.

Methods of the invention may further comprise screening for test compounds, such as small organic molecule, antibodies, nucleic acids or peptides, which bind to the same interaction site on a target protein as a biomolecule identified as described above.

Conventional techniques, such as displacement assays may be employed, to screen for compounds which compete with the biomolecule for binding to the target protein. For example, a method may comprise contacting a complex comprising the target protein bound to the biomolecule with a test compound. Displacement of the biomolecule by the test compound is indicative that test compound binds to the target protein at the same site as the phylomer. Standard displacement assay platforms, such as Alpha-LISA™ or fluorescence polarisation, may be employed.

Further displacement assays may be performed using truncated versions of the biomolecule in order to determine the key binding determinants in the binding interface. High throughput small molecule displacement screens may then be performed using a fluorescently-labelled version of these ‘minimised’ biomolecules.

Small molecules which can displace the test compound from the target protein are predicted to also inhibit the activity of the target protein in a cell, and may be useful in the development of therapeutics.

Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and tables described below.

### EXAMPLES

### Example 1

In order to tune the levels of miRNA and reporter proteins in the screening system to generate an appropriate signal window, a number of approaches were adopted. The presence of residual reporter protein in the screening system, which may have remained in the cells for some time after it has been translated, can mask the detection of mCherry reporter protein and so give sub-optimal results. The following studies were carried out to optimise the detection of the mRNA and reporter protein so that the read-out is enhanced and the method is able to detect signals to a significantly higher degree of accuracy.

A representative list of vectors we have used to address these issues is shown in Table 1 below.

TABLE 1

| Vector | | | |
|---|---|---|---|
| number | Vector name | Promoter | Destabilisation |
| 192 | pmC4.10CMV | CMV | No destabilisation |
| 193 | pmC4.11CMV | CMV | PEST destabilisation |
| | | | sequence |
| 194 | pmC4.22CMV | CMV | CL1 and PEST |
| | | | destabilisation |
| | | | sequences |
| 195 | pmC4.11HSV-TK | HSV-TK | PEST destabilisation |
| | | | sequence |
| 196 | pmC4.22HSV-TK | HSV-TK | CL1 and PEST |
| | | | destabilisation |
| | | | sequences |
| 197 | pmC4.10CMV-PURO | CMV | No destabilisation |
| 198 | pmC4.11CMV-PURO | CMV | PEST destabilisation |
| | | | sequence |
| 199 | pmC4.11CMV-TK-PURO | HSV-TK | PEST destabilisation |
| | | | sequence |

In this series of vectors, mCherry expression levels were regulated by using promoters of differing strengths. For example the human cytomegalovirus (CMV) promoter or the HSV-TK promoter. Further regulation of reporter protein expression has been achieved by including an in-frame destabilization sequence, for example a PEST sequence, within the coding sequence of the reporter.

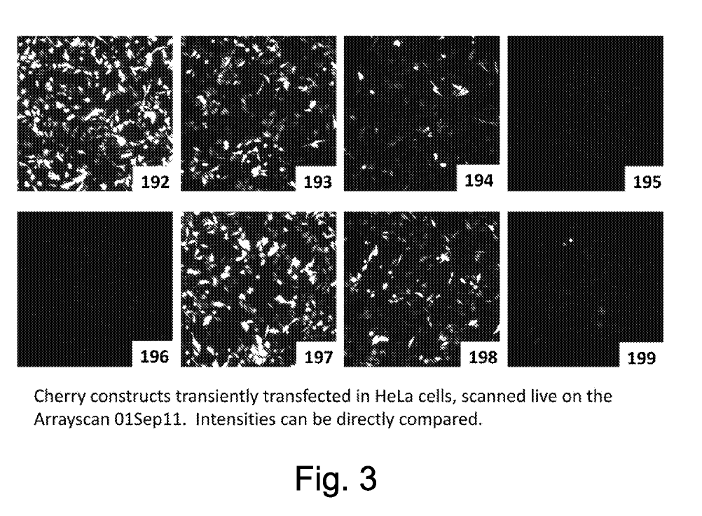

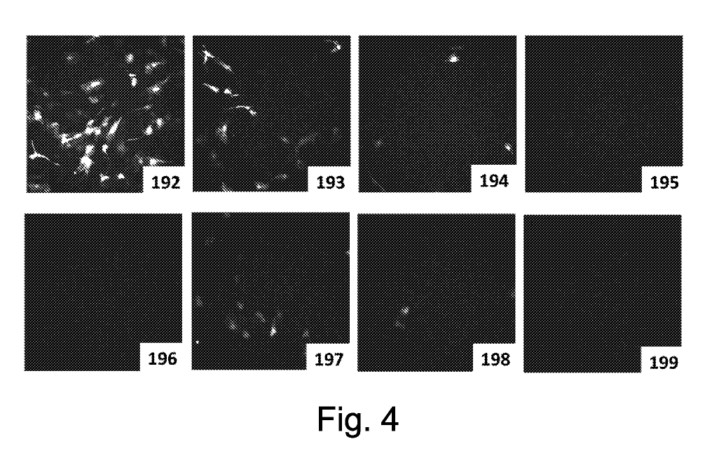

Reporters were further down-regulated by the inclusion of a CL1 degron sequence at the C-terminal, a sequence which specifically targets proteins for proteosomal degradation. As shown in FIGS. 3 and 4, by transfection of vectors into two different cell types, these different approaches lead to very different levels of reporter expression.

The results show that a first nucleic acid comprising a PEST sequence within the coding sequence of the reporter leads to significant decrease of expression. A combination of PEST and CL1 degron at the C-terminus of the coding sequence provides a further reduction of expression, thereby improving the sensitivity and efficiency of the method described herein.

### Example 2

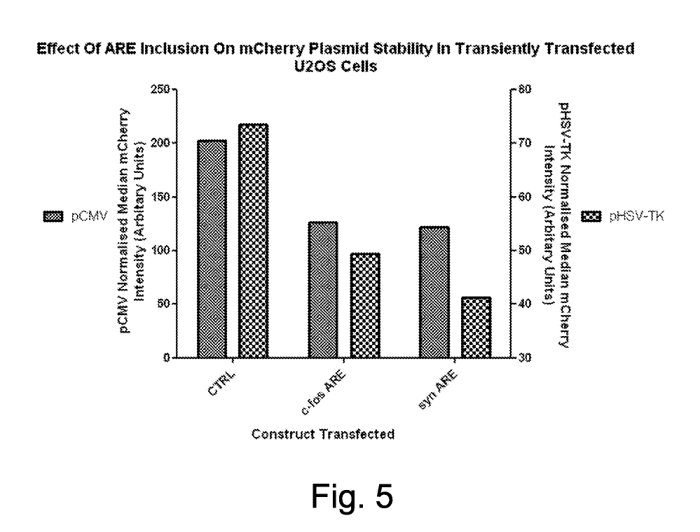

AU-Rich Elements (ARES) were incorporated into the 3′ UTR of its mRNA. FIG. 5 shows a comparison between mCherry reporters engineered to be unmodified (CTRL), to contain a naturally-occurring ARE (C-fos) or to contain a synthetic (Syn) ARE. The data shows the reduction in mCherry signal upon inclusion of an ARE using either the CMV or HSV-TK promoters to drive expression.

By using various combinations of promoters, destabilization sequences and mRNA degradation sequences, we can “fine-tune” the levels of miRNA and reporter protein so that they are appropriate for use in the screening system.

Sequences

SEQ ID No: 1
5′ TTGATGTTGACGTTGTAGGCGGTTTTGGCCACTGACTGACCGCCTA
CAGTCAACATCAA 3′

1. A method of screening for a test compound which inhibits a cell signalling pathway comprising:
(1) providing a population of mammalian cells having an active cell signalling pathway, each mammalian cell comprising:
(a) a first heterologous nucleic acid comprising;
(i) a nucleotide sequence encoding a first detectable reporter and,
(ii) a constitutive regulatory element which is operably linked to the nucleotide sequence; and,
(b) a second heterologous nucleic acid comprising:
(i) a nucleotide sequence encoding a repressor molecule which inhibits, inactivates or suppresses expression of the first detectable reporter,
(ii) a nucleotide sequence encoding a second detectable reporter; and,
(iii) a signal-activated regulatory element which is activated by said cell signalling pathway,
said signal-activated regulatory element being operably linked to the nucleotide sequence encoding the repressor molecule and the nucleotide sequence encoding the second detectable reporter,
(2) introducing a library of test compounds into said population of mammalian cells, and;
(3) determining the expression of the first and the second detectable reporters in one or more of the population of transfected cells,
wherein expression of the first detectable reporter but not the second detectable reporter in a transfected cell is indicative that a test compound from the library inhibits said cell signalling pathway.

2. A method according to claim 1 comprising;
(4) identifying one or more cells in the population which express the first detectable reporter but not the second detectable reporter, wherein a test compound from the library introduced into the one or more cells is a putative inhibitor of the cell signalling pathway.

3. A method according to claim 1 wherein the cell signalling pathway is constitutively activated in the mammalian cell.

4. A method according to claim 1, wherein the population of mammalian cells is provided by transfecting a population of mammalian cells with the first and second heterologous nucleic acids.

5. (canceled)

6. A method according to claim 1, wherein the repressor molecule is a miRNA.

7. A method according to claim 1, wherein the first and second detectable reporters are fluorescent proteins, and wherein the expression of the first and second detectable reporters is determined by measuring the fluorescent emission of the reporters.

8. A method according to claim 7 wherein the first and second detectable reporters are Cherry and emGFP respectively.

9. (canceled)

10. A method according to claim 1, wherein the test compounds are biomolecules, and wherein the library of biomolecules is introduced into the population of mammalian cells by expressing a library of nucleic acids encoding a diverse population of test biomolecules in said population.

11. (canceled)

12. A method according to claim 10, comprising, before step (2), transfecting the population of mammalian cells with the library of nucleic acids encoding the population of test biomolecules.

13. (canceled)

14. A method according to claim 10, wherein the test biomolecules are peptides.

15. A method according to claim 14 wherein the test biomolecules are phylomers.

16. A method according to claim 10, comprising isolating one or more mammalian cells which express the first detectable reporter but not the second detectable reporter.

17. A method according to claim 16 wherein cells are isolated by fluorescence activated cell sorting.

18. (canceled)

19. A method according to claim 16, comprising amplifying, cloning, isolating, and/or sequencing the nucleic acid encoding the test biomolecule or biomolecules from said one or more mammalian cells which express the first detectable reporter but not the second detectable reporter.

20. (canceled)

21. A method according to claim 19 comprising (5) providing a further population of mammalian cells comprising:
(a) a first heterologous nucleic acid comprising;
(i) a nucleotide sequence encoding a first detectable reporter and,
(ii) a constitutive regulatory element which is operably linked to the nucleotide sequence; and,
(b) a second heterologous nucleic acid comprising:
(i) a nucleotide sequence encoding a repressor molecule which inhibits, inactivates or suppresses expression of the first detectable reporter,
(ii) a nucleotide sequence encoding a second detectable reporter; and,
(iii) a signal-activated regulatory element which is activated by said cell signalling pathway, said signal-activated regulatory element being operably linked to the nucleotide sequence encoding the repressor molecule and the nucleotide sequence encoding the second detectable reporter,
(6) transfecting said further population of cells with said population of nucleic acids encoding test biomolecules isolated from the one or more mammalian cells positive for the first detectable reporter and negative for the second detectable reporter,
(7) expressing the population of nucleic acids in the further population of said mammalian cells, and
(8) determining the expression of the first and the second detectable reporters in the mammalian cells,
wherein expression of the first detectable reporter but not the second detectable reporter is indicative that the test biomolecule expressed by a nucleic acid in a mammalian cell is an inhibitor of said cell signalling pathway,
(9) identifying one or more mammalian cells in the further population which express the first detectable reporter but not the second detectable reporter, and;
(10) isolating the one or more mammalian cells which express the first detectable reporter but not the second detectable reporter.

22. A method according to claim 21 comprising isolating nucleic acids encoding test biomolecules from the one or more mammalian cells isolated in step (10).

23. (canceled)

24. A method according to claim 10 comprising expressing a nucleic acid isolated from a mammalian cell which expresses the first detectable reporter but not the second detectable reporter to produce the test biomolecule.

25. A method of identifying a protein, protein region or protein:protein interaction (PPI) site which may be a useful target for the therapeutic modulation of a cell signalling pathway, the method comprising:
providing a test compound which causes a mammalian cell to express the first detectable reporter but not the second detectable reporter in a method according to claim 1,
identifying an intracellular binding partner which binds the test biomolecule, said binding partner being a candidate target protein for modulation of a cell signalling pathway.

26. A method according to claim 25 wherein the test compound is a biomolecule which is encoded by a nucleic acid from a mammalian cell.

27. A method according to claim 26 comprising identifying a region of the intracellular binding partner which binds to the test compound, said region being a candidate target region or site for modulation of a cell signalling pathway.

28. (canceled)

29. A mammalian cell or a population of mammalian cells, each cell comprising:
(a) a first heterologous nucleic acid comprising;
(i) a nucleotide sequence encoding a first detectable reporter and,
(ii) a constitutive regulatory element which is operably linked to the nucleotide sequence; and,
(b) a second heterologous nucleic acid comprising:
(i) a nucleotide sequence encoding a repressor molecule which inhibits, inactivates or suppresses expression of the first detectable reporter,
(ii) a nucleotide sequence encoding a second detectable reporter; and,
(iii) a signal-activated regulatory element which is activated by said cell signalling pathway,
said signal-activated regulatory element being operably linked to the nucleotide sequence encoding the repressor molecule and the nucleotide sequence which encodes the second detectable reporter.

30. A cell population according to claim 29 which is transfected with a library of nucleic acids encoding a diverse population of test biomolecules.

31. A cell or population according to claim 29 wherein the repressor molecule is miRNA.

32. A vector or combination of vectors which comprises:
(a) a first heterologous nucleic acid comprising;
(i) a nucleotide sequence encoding a first detectable reporter and,
(ii) a constitutive regulatory element which is operably linked to the nucleotide sequence; and,
(b) a second heterologous nucleic acid comprising:
(i) a nucleotide sequence encoding an repressor molecule which inhibits, inactivates or suppresses expression of the first detectable reporter,
(ii) a nucleotide sequence encoding a second detectable reporter; and,
(iii) a signal-activated regulatory element which is activated by said cell signalling pathway,
said signal-activated regulatory element being operably linked to the nucleotide sequence encoding the repressor molecule and the nucleotide sequence which encodes the second detectable reporter.

33. A vector or combination according to claim 32 wherein the repressor molecule is miRNA.


SEQUENCE LISTING (2 sequences)

SEQ ID NO: 1 - DNA, 59 bp, Artificial Sequence
  Synthetic polynucleotide
  misc_feature (12)..(13): modified
ttgatgttgacgttgtaggcggttttggccactgactgaccgcctacagtcaacatcaa

SEQ ID NO: 2 - DNA, 10 bp, Artificial Sequence
  Synthetic polynucleotide
agatcaaagg